(19) United States
(12) Patent Application Publication
(10) Pub. No.: US 2024/0319186 A1
(43) Pub. Date: Sep. 26, 2024

(54) NANOWIRE-BASED IMMUNOFLUORESCENCE KIT FOR DETECTING SARS CORONAVIRUS 2 ANTIBODY, AND USE THEREOF

(71) Applicant: KOREA RESEARCH INSTITUTE OF CHEMICAL TECHNOLOGY, Daejeon (KR)

(72) Inventors: Hong Gi KIM, Daejeon (KR); Jung KIM, Daejeon (KR); Bum Tae KIM, Daejeon (KR); Jonghwan LEE, Daejeon (KR); Nam Hoon KIM, Daejeon (KR); Sung Kyun LEE, Daejeon (KR)

(21) Appl. No.: 18/271,640

(22) PCT Filed: Jan. 11, 2022
(86) PCT No.: PCT/KR2022/000405

§ 371 Date: Jul. 10, 2023

(30) Foreign Application Priority Data
Jan. 11, 2021 (KR) 10-2021-0003195

(51) Int. Cl. G01N 33/569; G01N 21/64; G01N 33/531; G01N 33/553

(52) U.S. Cl. CPC G01N 33/56983; G01N 21/6428; G01N 33/531; G01N 33/553; G01N 2021/6439; G01N 2333/165; G01N 2469/20

(57) ABSTRACT

The present invention relates to a nanowire-based immunofluorescence kit for detecting a SARS coronavirus 2 antibody, and a use thereof, and more specifically, to a zinc oxide nanowire-based immunofluorescence kit for detecting a SARS coronavirus 2 antibody, and a use thereof, the kit comprising a step of growing nanowires on a substrate, introducing a functional group on the grown nanowires, and fixing a SARS coronavirus 2 antigen. The nanowire array of the present invention has enhanced detection sensitivity to enable more accurate diagnosis of whether or not a SARS coronavirus 2 antibody is produced.

### TECHNICAL FIELD

The present invention relates to a nanowire-based immunofluorescence kit for detecting a SARS coronavirus 2 antibody, and a use thereof.

### BACKGROUND ART

Fluorescence immunoassay is an analysis method for detecting an antigen-antibody reaction by using fluorescence, and the fluorescence can be applied to various fields such as photonics, molecular biology, material science, and chemistry, as well as medical, pharmaceutical, and genetic fields including in vivo drug and protein tracking, biomolecule detection, and the like.

Although the fluorescence analysis method provides high detection sensitivity, there are problems such as limitation of quantum efficiency due to indirect labeling, extinction phenomenon due to intermolecular aggregation, superposition phenomenon due to the fluorescence of materials themselves other than fluorescent molecules, and fluorescent signal stability. The fluorescence intensity of fluorescent molecules is a major factor that determines the performance of fluorescence-based technology, and nanomaterial utilization technology is being studied for fluorescence signal improvement.

Recently, nanomaterials have various forms, such as nanoparticles, nanowires, and nanotubes, and have been used in various technical fields by using unique physical, chemical, optical and electrical characteristics thereof. It is possible to adjust various characteristics of a nanostructure according to the composition, shape or arrangement of the nanoparticles. Among them, nanowires, which are one-dimensional nanomaterials, have a diameter of less than 10 nm to several hundred nm, have excellent crystallinity, and have characteristics such as high chemical reactivity, quantum confinement effect, self-assembly, and stress relaxation due to a large specific surface area. Nanowires can be synthesized based on various materials such as Si, ZnO, GaN, and SnO₂, and among them, zinc oxide (ZnO) nanowires are attracting attention due to the excellent photoelectric characteristics, biocompatibility, low toxicity, and easy surface modification characteristics.

When the nanowires are used for a bio-device, a large surface area for fixing biomolecules is provided, thereby improving detection signals according to an immune response of the biomolecules and achieving the high sensitivity. Accordingly, technologies for improving fluorescence signals through accurate fixation of biomolecules on the nanostructure have been developed. For example, Korean Patent No. 10-1837827 discloses a biochemical molecule detection apparatus comprising: a substrate; one or more nanostructures fixed to the substrate and vertically grown from the substrate; and an aptamer complex fixed to the surface of the nanostructures, selectively binding to a biochemical molecule, and including at least one double-helical DNA in which fluorescence staining agents that generate fluorescence are intercalated, which can increase the binding efficiency of the biochemical molecules by controlling the characteristics of the nanostructures. In addition, Korean Patent No. 10-1163535 maximized the ratio of three-dimensional volume to surface area by exposing nanowires on a nanotemplate to connect bio-nanoparticles and arranging antibodies on the nanosurface with the controlled orientation by physical interaction.

Meanwhile, COVID-19 is classified as a class 1 infectious disease under the novel infectious disease syndrome and SARS coronavirus 2 is an RNA virus belonging to Coronaviridae. COVID-19 has been spread through droplets (saliva) and contact so far, in particular, through droplets (saliva) produced when coughing or sneezing, and it is known that COVID-19 can be spread when a person touches eyes, nose, and mouth after touching objects contaminated with SARS coronavirus 2. It is usually known as an incubation period is 1 to 14 days, and an average incubation period is 4 to 7 days. Major symptoms include various respiratory infections from mild to severe symptoms such as fever, feebleness, cough, dyspnea, and pneumonia, and further include phlegm, sore throat, headache, hemoptysis, nausea, diarrhea, and the like and in order to treat these, symptomatic treatment such as fluid supplementation and antipyretic drugs and general-purpose antiviral agents are administered in clinical practice, but there are no specific antiviral agents. In order to diagnose the infection of SARS coronavirus 2, the virus is isolated from the upper or lower respiratory tract samples, and infection is diagnosed through real-time gene amplification of specific genes.

Existing virus detection uses molecular biological methods such as PCR (gene amplification), the PCR technique has high sensitivity because it is amplified exponentially in proportion to time, but it is difficult to apply the PCR technique to the field because it requires specialized pre-treatment and requires complex and specialized experiments and equipment. Currently, virus infection is diagnosed through real-time RT-PCR using primers and/or probes that specifically bind to cDNA obtained by extracting viral RNA from a sample from a patient infected with SARS coronavirus 2 in the laboratory and reverse transcribed to DNA. However, such real-time gene amplification has a disadvantage in that it is difficult to utilize it for rapid point-of-care.

In the case of the antigen-antibody-based MERS in vitro diagnostic technology, a product capable of point-of-care based on immunochromatography using a sandwich ELISA technique and a product using an indirect ELISA technique by using a recombinant protein antigen were developed have been developed, and a virus diagnostic technology through antibody detection based on immunofluorescence method and protein microarray has been studied. Antigen-antibody-based diagnostic technology has the advantage that the diagnostic time is much shorter and the specificity is superior compared to the gene detection-based technology, but there is a problem of low sensitivity, and thus there is a need to improve it.

Accordingly, in the present invention, it was to develop a nanowire-based immunofluorescence kit for detecting SARS coronavirus 2 antibodies.

### DISCLOSURE OF THE INVENTION

### Technical Problem

In order to solve the above problems, the purpose of the present invention is to provide a nanowire-based immunofluorescence kit and a use thereof for detecting SARS coronavirus 2 antibodies, and a SARS coronavirus 2 detection effect is confirmed by using a nanowire array including antigens immobilized on the surface of nanowires, thereby completing the present invention.

### Technical Solution

In order to solve the above problems, the present invention provides a kit for detecting a SARS coronavirus 2 antibody, the kit including: a substrate; nanowires grown on the substrate; and SARS coronavirus 2 antigens immobilized on surfaces of the nanowires.

In one embodiment of the present invention, the SARS coronavirus 2 antigens may be nucleocapsid or spike protein antigens, and the kit may include an antibody to which a fluorescence marker recognizing the SARS coronavirus 2 antibody is bound.

In another embodiment of the present invention, the substrate may be any one selected from the group consisting of glass, silicon wafer, polystyrene, and polyethylene, the nanowires may be zinc oxides, the nanowires may include 0.01 M zinc acetate dihydrate and 0.03 M sodium hydroxide as a seed solution, the nanowires may include 25 mM zinc nitrate hexahydrate, 25 mM hexamethylene tetramine, and 5 mM polyethyleneimine as a precursor solution, and the surfaces of the nanowires may be treated with 4 vol % of 3-aminopropyltriethoxysilane and 2 vol % of glutaraldehyde to form a functional group.

In still another embodiment of the present invention, the SARS coronavirus 2 antibody may be detected from one or more isolated samples selected from the group consisting of whole blood, serum, and plasma.

In addition, the present invention provides a composition for diagnosing a SARS coronavirus 2 antibody, the composition including: a substrate; nanowires grown on the substrate; and SARS coronavirus 2 antigens immobilized on the surfaces of the nanowires.

Further, the present invention provides a method for manufacturing a nanowire array for detecting a SARS coronavirus 2 antibody, the method comprising: (a) preparing a nanowire seed solution; (b) preparing a nanowire precursor solution; (c) growing nanowires on a substrate; (d) introducing functional groups into the nanowires; and (e) immobilizing SARS coronavirus 2 antigens to the functional groups.

Also, the present invention provides a nanowire array for detecting a SARS coronavirus 2 antibody, the nanowire array being manufactured by: (a) preparing a nanowire seed solution; (b) preparing a nanowire precursor solution; (c) growing nanowires on a substrate; (d) introducing functional groups into the nanowires; and (e) immobilizing SARS coronavirus 2 antigens to the functional groups.

### Advantageous Effects

The present invention relates to a nanowire-based immunofluorescence kit for detecting a SARS coronavirus 2 antibody and a use thereof, and may be useful for antibody detection and antibody diagnosis tests for individual antigens produced from an individual infected with SARS coronavirus 2.

### BRIEF DESCRIPTION OF THE DRAWINGS

FIG. 1 illustrates a schematic diagram of a nanowire-based immunofluorescence kit for detecting a SARS coronavirus 2 antibody according to an embodiment of the present invention.

FIG. 2 shows a photograph of a nanowire array according to an embodiment of the present invention.

FIG. 3 shows a measurement result of an SEM image of nanowires according to an embodiment of the present invention.

FIG. 4 shows a measurement result of fluorescence signals depending on a nanowire array synthesis time (left) or GFP concentration (right) according to an embodiment of the present invention.

FIG. 5 shows a measurement result of fluorescence signals (left) of a nanowire array coated with a nucleocapsid according to an embodiment of the present invention and obtained by measuring an antibody through an immune reaction, and a fluorescence image (right).

FIG. 6 shows a measurement result of fluorescence signals of a nanowire array coated with a spike antigen according to an embodiment of the present invention and obtained by measuring an antibody through an immune reaction.

### MODE FOR CARRYING OUT THE INVENTION

Hereinafter, a preferred embodiment of the present invention will be described in detail. Also, several specific details such as specific constituents are described in the following description. However, they are provided only to help more general understanding of the present invention. It will be obvious to a person skilled in the art that the invention can be made without such the specific details. In addition, in the following description of the present invention, detailed descriptions related to well-known functions or configurations will be ruled out in order not to unnecessarily obscure subject matters of the present invention.

The present invention provides a kit for diagnosing a SARS coronavirus 2 antibody, the composition including: a substrate; nanowires grown on the substrate; and SARS coronavirus 2 antigens immobilized on the surfaces of the nanowires.

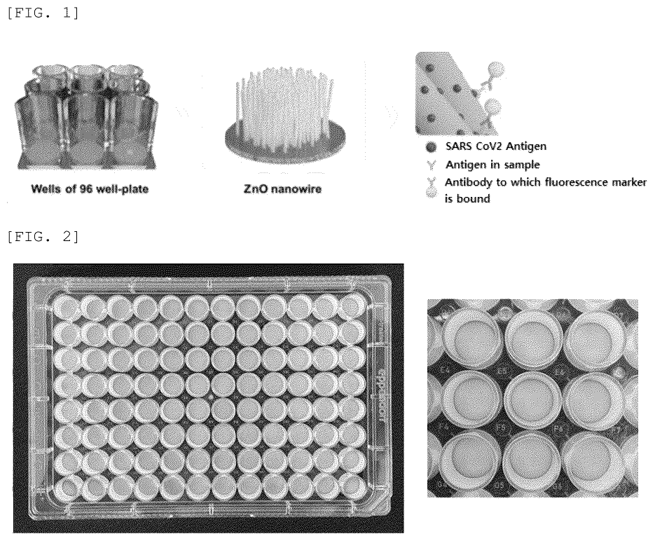

FIG. 1 is a schematic diagram of a kit for detecting a SARS coronavirus 2 antibody of the present invention, the kit including a substrate on which nanowires are grown, SARS coronavirus 2 antigens immobilized on the nanowires, antibodies in a sample that react with the antigens, and an antibody to which a fluorescence marker recognizing the antibodies is bound.

The SARS coronavirus 2 antigen or antibody means all antigens present on SARS coronavirus 2 or all antibodies capable of binding thereto, and preferably, a nucleocapsid or spike protein antigen or an antibody capable of binding thereto, but is not limited thereto.

In addition, the term “SARS coronavirus 2 antibody” as used herein may be generally used in the same concept as a primary antibody used in the assay using an immune response.

The SARS coronavirus 2 antigens may be immobilized on the nanowires through linkers serving as an intermediate bridge. Random immobilization without considering the orientation of the antigen may be accompanied by the deterioration in the performance of target detection because the random immobilization may not be used in the antigen-antibody binding or the antigen-antibody binding performance is deteriorated when the antibody and the binding site are not exposed to the outside even though the amount of immobilized antigens is large. The linker is for site-specifically immobilizing the antigens on the nanowires by introducing the functional groups to the nanowires, and is preferably 3-aminopropyltriethoxysilane and glutaraldehyde, and more preferably is treated with 4 vol % of 3-aminopropyltriethoxysilane and 2 vol % of glutaraldehyde, but is not limited thereto.

In the present invention, the antibody used to detect the SARS coronavirus 2 antibody is an antibody capable of specifically binding to all kinds of antibodies that bind to the SARS coronavirus 2 antigen, and may be used in the same concept as a secondary antibody used in a test method using an immune response in the present specification.

In addition, as the probe for measuring whether or not antibodies bind, all means known in the art may be used, but in consideration of ease of analysis, and the like, in the present invention, the form in which a fluorescent marker is bound is preferably used. Examples of the fluorescence marker include FAM, VIC, TAMRA, JOE, ROX, HEX, Cy3, Cy5, Texas Red, and the like.

In the present invention, the antibody for detecting the SARS coronavirus 2 antibody is preferably an antibody to which the fluorescence marker is bound, and does not affect the immune response of the antibody, and may improve sensitivity by amplifying a signal, thereby enabling more accurate detection, but is not limited thereto.

Specifically, in the SARS coronavirus 2 antibody detection mechanism, an isolated sample such as blood is reacted with antigens immobilized on nanowires grown on the substrate, and an antibody in the isolated sample specifically bound to the antigen is reacted with a fluorescence-labeled antibody capable of specifically recognizing the antibody in the sample, thus detecting a fluorescence signal, thereby finally detecting whether the SARS coronavirus 2 antibody is present.

In the present invention, the substrate may be any one selected from the group consisting of glass, silicon wafer, polystyrene, and polyethylene, but is not limited thereto.

The nanowire is preferably zinc oxide, and the zinc oxide has a very stable polar surface, so that it is easy to form several nanostructures. When the nanowires are grown on the substrate, a large surface area for immobilization of the antibodies is provided, and thus the substrate is efficiently used as a substrate for an antigen-antibody immune response. On the other hand, when 0-dimensional nanoparticles are used instead of the nanowires, it may be difficult to achieve an accurate signal enhancement effect or reproducibility due to a wide size distribution of nanoparticles and a non-uniform distribution on the substrate. In addition, in the case of the two-dimensional plate, the degree of freedom of the antibodies decreases, and thus the antibody activity may deteriorate.

A seed solution for forming a zinc oxide seed layer may include zinc acetate dihydrate, preferably, 0.01 M zinc acetate dihydrate and 0.03 M sodium hydroxide, but is not limited thereto.

The zinc precursor solution may include zinc nitrate hexahydrate, hexamethylene tetramine, and polyethyleneimine. The hexamethylene tetramine and the polyethyleneimine make the zinc precursor grow in a specific plane direction (one-dimensional direction) on the substrate when the zinc precursor is converted into zinc oxide, and affect the morphological characteristics of the nanowires, and specifically, may serve to promote or suppress the growth of a specific crystal plane. That is, it serves to suppress the growth in the lateral direction so that the zinc precursor is converted into an oxide and grows into a one-dimensional structure of the nanowires. In this case, the hexamethylene tetramine functions as a reducing agent, and polyethyleneimine induces the formation of an array having a one-dimensional structure. Preferably, the precursor solution of the nanowires includes 25 mM zinc nitrate hexahydrate, 25 mM hexamethylene tetramine, and 5 mM polyethyleneimine, but is not limited thereto.

In the present invention, the SARS coronavirus 2 antibodies may be detected in all isolated samples in which the antibodies are present, and preferably, they may be detected from one or more isolated samples selected from the group consisting of whole blood, serum, and plasma, but is not limited thereto.

In addition, the present invention provides a composition for diagnosing a SARS coronavirus 2 antibody, the composition including: a substrate; nanowires grown on the substrate; and SARS coronavirus 2 antigens immobilized on the surfaces of the nanowires.

Further, the present invention provides a method for manufacturing a nanowire array for detecting a SARS coronavirus 2 antibody, the method comprising: (a) preparing a nanowire seed solution; (b) preparing a nanowire precursor solution; (c) growing nanowires on a substrate; (d) introducing functional groups into the nanowires; and (e) immobilizing SARS coronavirus 2 antigens to the functional groups.

Also, the present invention provides a nanowire array for detecting a SARS coronavirus 2 antibody, the nanowire array being manufactured by: (a) preparing a nanowire seed solution; (b) preparing a nanowire precursor solution; (c) growing nanowires on a substrate; (d) introducing functional groups into the nanowires; and (e) immobilizing SARS coronavirus 2 antigens to the functional groups.

The diameter of the nanowires is preferably 50 nm to 100 nm.

In step (c) above, the nanowires may be grown by using a hydrothermal synthesis method, and step (c) above includes: i) applying the zinc seed solution to the substrate to form a seed layer; and ii) dipping the substrate into the zinc precursor solution by facing the seed layer down and reacting the substrate at 95° C. In this case, the length of the nanowires may be adjusted by adjusting the reaction time between the zinc oxide seed layer and the zinc precursor solution.

Step (d) above includes: i) immersing the substrate on which the nanowires are grown into a 3-aminopropyltriethoxysilane solution to react the substrate at room temperature for 2 hours; and ii) immersing the substrate into a glutaraldehyde solution to react the substrate at 4° C. for 2 hours. The surfaces of the nanowires are modified with 3-aminopropylethoxysilane, reacted with glutaraldehyde, and then bound to the SARS coronavirus 2 antigens, thereby easily immobilizing the antigens and improving the immobilizing force.

In step (e) above, it is preferable that the SARS coronavirus 2 antigens are cultured and immobilized at room temperature for 1 hour on the substrate to which the functional group is attached, but the present invention is not limited thereto.

Advantages and features of the present invention, and implementation methods thereof will be clarified with reference to the embodiments which will be described below in detail. The present invention may, however, be embodied in different forms and should not be construed as limited to the embodiments set forth herein. Rather, these embodiments are provided so that this disclosure will be thorough and complete, and will fully convey the scope of the present disclosure to those skilled in the art. Further, the present disclosure is only defined by scopes of claims.

### <Example 1> Preparation of Nanowire Array

### 1-1. Nanowire Seed Solution Preparation

A solution of 0.01 M zinc acetate dihydrate in methanol (Zn(CH₃COO)₂·2H₂O) and a solution of 0.03 M sodium hydroxide in methanol were heated at 60° C., respectively, and then the solution of zinc acetate dihydrate was slowly dropped into the solution of sodium hydroxide drop-by-drop and stirred at 200 rpm. Then, the mixture was heated while being stirred until the volume of the mixture became half.

### 1-2. Nanowire Precursor Solution Preparation

Zinc nitrate hexahydrate (Zn(NO₃)₂·6H₂O), hexamethylene tetramine (C₆H₁₂N₄), and polyethyleneimine (H(NHCH₂CH₂)ₙNH₂) were each added to deionized water and stirred at 200 rpm to prepare precursor solutions of 25 mM zinc nitrate hexahydrate, 25 mM hexamethylene tetramine, and 5 mM polyethyleneimine in deionized water.

### 1-3. Nanowire Synthesis

The seed solution was dropped and thinly applied to the substrate, then washed with ethanol before being completely dried, and dried with nitrogen using an air gun, and this process was repeated three times. Thereafter, the substrate was heated at 350° C. for 5 minutes using a hot plate to form a seed layer. A polyimide tape with the desired pattern was attached to the completely cooled substrate to expose only the part in which the nanowires were synthesized. The surface of the substrate was placed in a container while facing down, the precursor solution was filled so that the substrate was completely immersed, and then this was placed in a convection oven at 95° C. for 5 hours to synthesize nanowires. After 5 hours, the substrate on which the nanowires were synthesized was taken out, the tape was removed, the substrate was washed with deionized water, and dried with nitrogen. In this case, the nanowire synthesis time may vary with the desired length of the nanowires.

### 1-4. Formation of Functional Group for Immobilizing SARS Coronavirus 2 Antigens

The substrate on which the nanowires were synthesized was immersed in an ethanol-based 4 vol % 3-aminopropyltriethoxysilane solution and reacted at room temperature for 2 hours. After the reaction, the substrate was sufficiently washed with ethanol, sufficiently washed with deionized water, and then dried by blowing nitrogen using the air gun. Then, the substrate was immersed in a PBS-based 2 vol % glutaraldehyde solution and reacted at 4° C. for 2 hours. After the reaction, the substrate was sufficiently washed with deionized water, and then dried by blowing nitrogen using the air gun. L. Wang et. al., “surface plasmon resonance biosensor based on water-soluble ZnO—Au nanocomposites,” Analytica Chimica Acta, 2009, 653, 109-115 are referenced.

### 1-5. Immobilization of SARS Coronavirus 2 Antigens

Control samples (Maxisorp) were prepared by using nanowires grown for 3 hours without surface modification (functional group formation), and coating with 1,000 or 2,000 ng/mL of SARS coronavirus 2 antigens.

SARS coronavirus 2 nucleocapsid (1,000 or 2,000 mg/ml at 100 μl/well) or spike protein antigen (150 ng/ml) was cultured at room temperature (RT) for 1 hour and attached to the substrate on which the functional group was formed, and then washed three times with 0.05% Tween®20 Tris-buffered saline (TBS-T) washing solution. Blocking was performed by reacting at RT for 1 hour using 300 μl/well of Invitrogen blocking buffer. The substrate was washed three times with 0.05% TBS-T washing solution.

### <Example 2> Evaluation of Nanowire Diameter and Length Characteristics

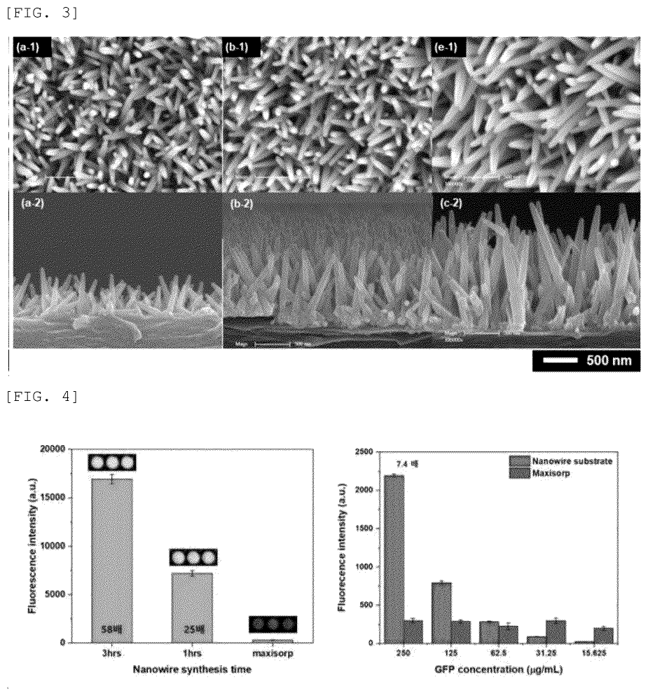

As shown in the SEM image of FIG. 3, the diameter of the nanowires synthesized by the hydrothermal synthesis method was 50 nm to 100 nm. The length of the nanowires was 2 μm when the nanowires were synthesized for 5 hours, and the length of the nanowires may be adjusted according to the synthesis time (about 500 nm/hr).

### <Example 3> Evaluation of Surface Area of Nanowire Substrate

In order to directly confirm the increase of the surface area, the GFP protein was immobilized on the substrate, to which the zinc oxide nanowires were applied according to the concentration, and the conventional substrate according to the concentration, and the protein immobilizing efficiency according to the increase of the surface area was confirmed by using a fluorescence signal. When the nanowires were synthesized for 1 hour or 3 hours, the fluorescence signal was increased by 25 times or 48 times compared to the conventional substrate, respectively.

As can be seen from the results of the above embodiment, it was observed that the conventional substrate was saturated at a GFP concentration of at least 15.626 μg/ml, but it was confirmed that the nanowire array substrate of the present invention had an increase in fluorescence density in a concentration-dependent manner, and the fluorescence density was increased by about 7 times or more at a GFP concentration of 250 μg/ml, thereby increasing the surface area compared to the conventional substrate (see FIG. 4).

### <Example 4> Detection of SARS Coronavirus 2 Nucleocapsid or Spike Protein Antibody

After the SARS coronavirus 2 nucleocapsid or spike protein antigens were attached on the nanowire substrate, in order to confirm the reactivity of the antibody, anti-NP polyclonal antibody or anti-spike monoclonal antibody having different concentrations (0.01 to 10,000 ng/mL) were reacted at room temperature for 1 hour to be attached thereto, and then the substrate was washed using the washing solution. A secondary antibody (Alexa 488 secondary antibody; ThermoFisher Scientific Inc.; 1 mg/ml thereof was diluted at 1:1000; 100 μl/well) to which fluorescence particles had been attached was attached to the substrate by reaction for 1 hour and then washed using the washing solution. In this case, Invitrogen Coating Buffer B and 0.1% skim milk in TBS-T were used as the antibody dilution solution.

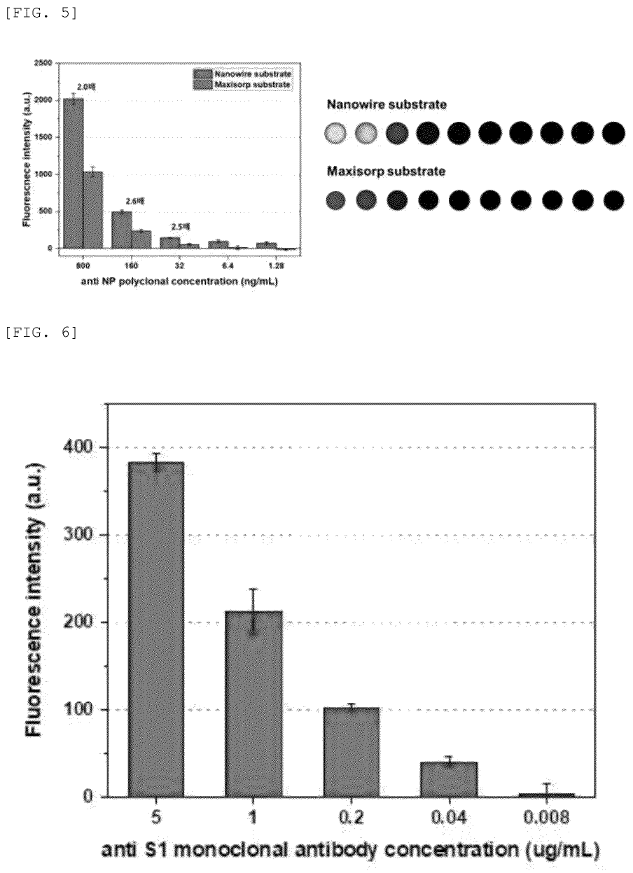

Anti-NP polyclonal antibodies having different concentrations (800, 160, 32, 6.4, and 1.28 ng/ml) were reacted, and then the secondary antibody to which a fluorescence marker had been attached was reacted, and then a fluorescence signal was observed using a fluorescence reader. As shown in FIG. 5, as a result of measuring the fluorescence signal intensity at each concentration, a significant fluorescence density was observed from 1.28 ng/ml, and it was possible to check a bright signal from 32 ng/ml concentration, and it may be seen that the sensitivity is excellent due to low detection limits.

As can be seen from the results of Examples above, the nanowire array for detecting an antibody against the SARS coronavirus 2 antigen of the present invention increases the immobilization force of the antibody by growing nanowires on the substrate, and accordingly, the signal sensitivity is increased by at least two times to exhibit a low detection limit of 32 ng/ml, and it is possible to detect the antibody against the SARS coronavirus 2 antigen with high sensitivity. In addition, since rapid diagnosis is also possible by using an antibody to which a fluorescence marker is bound, the superiority is secured in detecting the ability to produce the antibody against the SARS coronavirus 2 antigen.

In the case of spike antigens, anti-Si monoclonal antibodies having different concentrations (5,000, 1,000, 200, 40, and 8 ng/ml) were reacted on the substrate on which the nanowires coated with 150 ng/mL of the antigens were integrated, and then the secondary antibody to which the fluorescence marker previously used was attached was reacted to confirm the fluorescence signal. As shown in FIG. 6, it was confirmed that the fluorescence signal changes in proportion to the change in the concentration of the antibody. In fact, a polyclonal antibody against the spike antigen is formed in the blood of a patient infected with SARS coronavirus 2, and a more sensitive fluorescence signal may be detected when tested with the anti-Si polyclonal antibody in the future.

1. A kit for detecting a SARS coronavirus 2 antibody, the kit comprising:
a substrate;
nanowires grown on the substrate; and
SARS coronavirus 2 antigens immobilized on surfaces of the nanowires.

2. The kit of claim 1, wherein the SARS coronavirus 2 antigens are nucleocapsid or spike protein antigens.

3. The kit of claim 1, comprising an antibody to which a fluorescence marker recognizing the SARS coronavirus 2 antibody is bound.

4. The kit of claim 1, wherein the substrate is any one selected from the group consisting of glass, silicon wafer, polystyrene, and polyethylene.

5. The kit of claim 1, wherein the nanowires are zinc oxides.

6. The kit of claim 1, wherein the nanowires comprise, as a seed solution, 0.01 M zinc acetate dihydrate and 0.03 M sodium hydroxide.

7. The kit of claim 1, wherein the nanowires comprise, as a precursor solution, 25 mM zinc nitrate hexahydrate, 25 mM hexamethylene tetramine, and 5 mM polyethyleneimine.

8. The kit of claim 1, wherein the surfaces of the nanowires are treated with 4 vol % of 3-aminopropyltriethoxysilane and 2 vol % of glutaraldehyde to form functional groups.

9. The kit of claim 1, wherein the SARS coronavirus 2 antibody is detected from an isolated sample selected from the group consisting of whole blood, serum, and plasma.

10. A composition for diagnosing a SARS coronavirus 2 antibody, the composition comprising:
a substrate;
nanowires grown on the substrate; and
SARS coronavirus 2 antigens immobilized on surfaces of the nanowires.

11. A method for manufacturing a nanowire array for detecting a SARS coronavirus 2 antibody, the method comprising steps of:
(a) preparing a nanowire seed solution;
(b) preparing a nanowire precursor solution;
(c) growing nanowires on a substrate;
(d) introducing functional groups to the nanowires; and
(e) immobilizing SARS coronavirus 2 antigens to the functional groups.

12. A nanowire array for detecting a SARS coronavirus 2 antibody, the nanowire array being manufactured by steps of:
(a) preparing a nanowire seed solution;
(b) preparing a nanowire precursor solution;
(c) growing nanowires on a substrate;
(d) introducing functional groups to the nanowires; and
(e) immobilizing SARS coronavirus 2 antigens to the functional groups.
